(19) United States
(12) Patent Application Publication
(10) Pub. No.: US 2025/0327140 A1
(43) Pub. Date: Oct. 23, 2025

(54) A METHOD OF DETERMINING THE DOSE OF PSYCHEDELICS

(71) Applicants: MYND LIFE SCIENCES INC., Kelowna (CA); The University of British Columbia, Vancouver (CA)

(72) Inventor: Wilfred Jefferies, Surrey (CA)

(73) Assignees: MYND LIFE SCIENCES INC., Kelowna, BC (CA); The University of British Columbia, Vancouver, BC (CA)

(21) Appl. No.: 18/870,592

(22) PCT Filed: May 31, 2023
(86) PCT No.: PCT/CA2023/050743

§ 371 Date: Nov. 29, 2024

Related U.S. Application Data
(60) Provisional application No. 63/347,062, filed on May 31, 2022.

(51) Int. Cl. C12Q 1/6897; C12Q 1/6876; G01N 33/50; G01N 33/573

(52) U.S. Cl. CPC C12Q 1/6897; C12Q 1/6876; G01N 33/5023; G01N 33/573; C12Q 2600/136; C12Q 2600/158; G01N 2333/37

(57) ABSTRACT

The present invention provides a method of determining the amount of a psilocybin in a sample, the method comprising contacting a cell which expresses ABCF1 with the sample, comparing the expression of ABCF1 with a standard to determine the amount of psilocybinin in the sample. Also provided are methods of microdosing based on ABCF1 expression.

### FIELD OF THE INVENTION

The present invention relates to the field of therapeutics, in particular to determining the dose of psychedelics.

### BACKGROUND OF THE INVENTION

Microdosing of psychedelics has substantially increased in popularity for its cognitive benefits. Micro-dosing of psychedelics involves ingesting sub-hallucinogenic amounts of a psychedelic substance.

Psychedelic natural products such as mushrooms from the following genera: Agrocybe, Amanita, Conocybe, Galerina, Gymnopilus, Hypholoma, Inocybe, Panaeolus, Psilocybe, Pholiotina, Pluteus, and Weraroa or extracts thereof may contain varying concentrations of psychedelic compounds. As such, micro-dosing using such natural products may be challenging as the amount of psychedelic compounds in the product may vary. Accordingly, there is a need in the art to accurately determine the amount of psychedelic compounds in such products.

ATP-binding cassette sub-family F member 1 (ABCF1) has been associated with immune signaling and various autoimmune disorders. ABCF1 is an E2 ubiquitin-conjugating enzyme that regulates various innate immune responses in macrophages, including potentiation of TLR4 endocytosis and M2 polarization, and promotes endotoxin tolerance and survival during septic toxic shock (Arora et al., Immunity 50, 1-14, 2019).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a method of determining the dose of psychedelics. In certain aspects, there is provided a method of microdosing based on ABCF1 expression, the method comprising: a) determining ABCF1 expression in a sample obtained from a subject; b) administering a microdose of an agent (including extract) which modulates ABCF1 expression if ABCF1 expression is less than a pre-determined amount; c) obtaining a further sample from the subject after a pre-determined amount of time and determining ABCF1 expression; d) administering a microdose of said agent if ABCF1 expression is less than a pre-determined amount; optionally repeating steps c) and d) one or more times.

In another aspect, there is provided a method of psilocybin microdosing based on ABCF1 expression, the method comprising: a) determining ABCF1 expression in a sample obtained from a subject; b) administering a microdose of psilocybin if ABCF1 expression is less than a pre-determined amount; c) obtaining a further sample from the subject after a pre-determined amount of time and determining ABCF1 expression; d) administering a microdose of said psilocybin if ABCF1 expression is less than a pre-determined amount; optionally repeating steps c) and d) one or more times.

In another aspect, there is provided a method of determining the amount of a psychedelic compound which modulates ABCF1, such as psilocybin, in a sample, the method comprising contacting a cell which is capable of expressing ABCF1 or a reporter gene under the control of the ABCF1 promoter with the sample, comparing the expression of ABCF1 or reporter with a standard to determine the amount of the compound in the sample.

In another aspect, there is provided a method of determining the amount of a psilocybin in a sample, the method comprising contacting a cell which is capable of expressing ABCF1 or a reporter gene under the control of the ABCF1 promoter with the sample, comparing the expression of ABCF1 or reporter with a standard to determine the amount of psilocybin in the sample.

In another aspect, there is provided a method to identify agents that modulate ABCF1 expression, said method comprising contacting a cell expressing a reporter gene under the control of the ABCF1 promoter with the agent of interest; and measuring reporter gene product.

In another aspect, there is provided a method of determining the amount of a psilocybin in a sample, the method comprising contacting a cell which expresses ABCF1 with the sample, comparing the expression of ABCF1 with a standard to determine the amount of psilocybin in the sample, optionally the sample is a natural product or extract thereof.

### BRIEF DESCRIPTION OF THE FIGURES

These and other features of the invention will become more apparent in the following detailed description in which reference is made to the appended drawings.

FIG. 1 illustrates the effect of psylocibin, psylocin and their analogs on ABCF1 transcription in a macrophage cell line. ES=escitalopram; PSYB=Psylocibin; PSIC=Psilocin; DMT=4-Acetoxy-N, N-dimthyltryptamine; APF=O-Acetyl Psilocin Fumerate, and AOI=4-acetoxyindole.

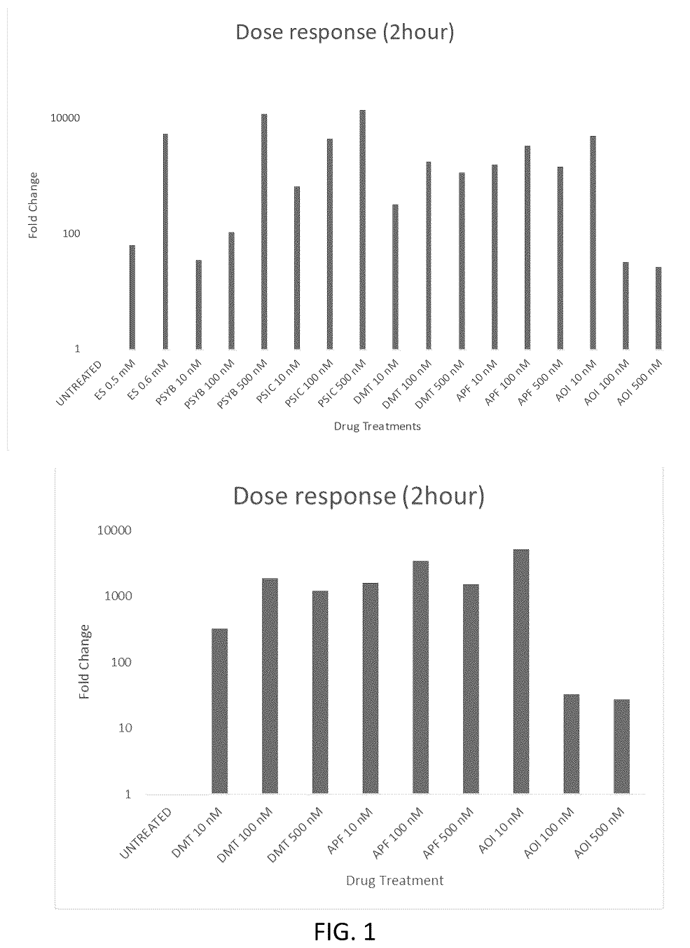

FIG. 2 illustrates ABCF1 expression in RAW cells treated with psilosybin.

FIG. 3 illustrates expression levels of Hif-α and ABCF1 2 hours post 500 nM psilosybin treatment.

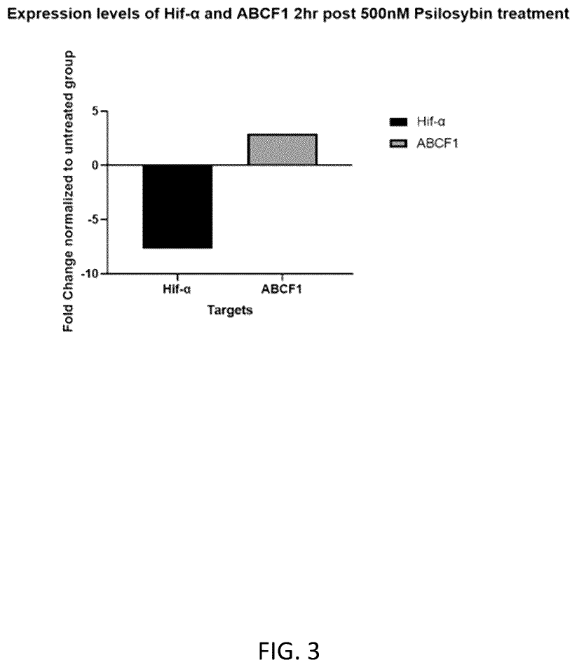

FIG. 4 illustrates ABCF1 expression in RAW cells treated for 2 hours, 24 hours and 26 hours.

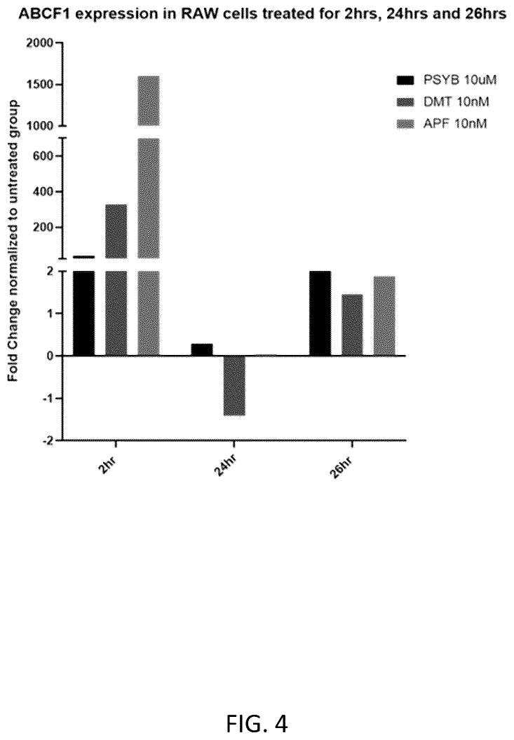

FIG. 5 illustrates dose response screening with different psychedelic drugs.

FIG. 6 illustrates ABCF1 expression indicating potential for microdosing.

FIG. 7 provides the plasmid map of an embodiment of an ABCF1 reporter gene construct comprising the sequence encoding EGFP under the control of the ABCF1 promoter.

FIG. 8 provides DC2.4 cells with and without ABCF1 GFP construct transfection. Medium GFP expression can be found in transfected DC2.4 cells, single sorted into a 96 well plate.

FIG. 9 provides results of drug screening in various clones of DC2.4 cells transfected with the ABCF1 GFP construct.

FIG. 10 demonstrates that the psylocybin analogue AOI upregulates ABCF1 after 1 hour drug treatment.

FIG. 11 demonstrates induction of ABCF1-EGFP-IRES with Esctalopram or AOI.

### DETAILED DESCRIPTION OF THE INVENTION

The present invention is based on the discovery that certain compounds including Selective serotonin re-uptake inhibitors (SSRIs) such as Escitalopram, psilocybins, analogs and derivatives thereof modulate the ABCF1 pathway. Accordingly, the present invention provides a method of measuring the amount of compounds which modulate the ABCF1 pathway, including but not limited to SSRIs, psilocybins in a substance/sample. In certain embodiments, the methods of the present invention allow for the dose of psilocybins and psilocybin-containing extracts and compounds to be accurately determined. In specific embodiments, the methods allow for microdoses to be accurately determined.

In certain embodiments, there is provided a method of microdosing based on ABCF1 expression. In such embodiments, the method comprises determining ABCF1 expression in a subject and administering a microdose of a compound the modulates ABCF1 based on the expression level. The steps of the method may be repeated at one or more times. A non-limiting illustrative embodiment is detailed below:
1. A sample is obtained from a subject. ABCF1 expression is determined.
2. A microdose of an agent (including extract) which modulates ABCF1 expression is administered if ABCF1 expression is less than a pre-determined amount. If ABCF1 expression is greater than the pre-determined amount, a microdose of the agent is not administered.
3. After a pre-determined amount of time, a further sample is obtained from the subject and ABCF1 expression is determined.
4. A microdose of the agent is administered if ABCF1 expression is less than a pre-determined amount. If ABCF1 expression is greater than the pre-determined amount, a microdose of the agent is not administered.
5. Steps 3 and 4 may be repeated on or more times.

In specific embodiments, there is provided a method of psilocybin or analogue thereof microdosing based on ABCF1 expression, the method comprising: a) determining ABCF1 expression in a sample obtained from a subject; b) administering a microdose of psilocybin or analogue thereof if ABCF1 expression is less than a pre-determined amount; c) obtaining a further sample from the subject after a pre-determined amount of time and determining ABCF1 expression; d) administering a microdose of said psilocybin or analogue thereof if ABCF1 expression is less than a pre-determined amount; optionally repeating steps c) and d) one or more times.

In certain embodiments, there is provided a method of determining the amount of a psychedelic compound known to modulate ABCF1 expression in a sample, the method comprising contacting a cell which is capable of expressing ABCF1 with the sample, comparing the expression of ABCF1 with a standard to determine the amount of psychedelic compound in the sample.

In specific embodiments, there is provided a method of determining the amount of a psilocybin or analogue thereof in a sample, the method comprising contacting a cell which is capable of expressing ABCF1 with the sample, comparing the expression of ABCF1 with a standard to determine the amount of psilocybin or analogue thereof in the sample.

Methods of measuring gene expression including mRNA and protein expression are known in the art. For example, mRNA may be measured using Northern blots, quantitative Reverse Transcription PCR (qRT-PCR) and microarrays. Protein expression may be measured using mass spectrometry including but not limited to SISCAPA (Stable Isotope Standards and Capture by Anti-Peptide Antibodies mass spectrometry (MS) and liquid-chromatography mass spectrometry (LC-MS) and immunoassays including but not limited to Enzyme-Linked Immunosorbent Assay (ELISA), Western-blotting, and immunoarrays.

There are secreted and cell retained forms of ABCF1. Accordingly, in certain embodiments, both forms are measured. In certain embodiments, secreted ABCF1 is measured. In certain embodiments, the cell retained form is measured.

In specific embodiments, a reporter gene is placed under the control of the ABCF1 promoter and the reporter gene product is measured.

In specific embodiments, the ABCF1 promoter comprises the following sequence or active fragment thereof:

(SEQ ID NO: 5)
CGTGTCTTCCTTGCCTACCAGCCTCACCTGATGGGCTCGTGTTCTCTCCG
TCCCCGATCCACTCGGGCTCCGGCAGCTGCTGCTTGGGCGCCTTCGGCAT
CGCGGTGGCAGAACTAGAAACGAGTTACAGATAGAAACTAGAATATGCTT
TTTAAAAAAACAAAAAACAAAACAAACAAAAAAACAGTATGCCTCAACTC
CTTCATACTAGTAGGAAATTATTATGTTCATTCCTTGAGTCTCGCGGCGT
CGGGAGGTCACGGCGTCAGGCTTCCCAGACAGTCGTAAACGCCATGTGTT
TACGCGACTGGAGCAAGCGGACGCCGGCCCCGCTCCGTCATTGCAGGCCA
CGCCTCCACTGAACCAGGGCCACGCCCCCGAGATGACGGCGAAGCTCGCA
CGTGCGCAGCCCGGGGGCGGGGTTGGCCGCGCCAGCTTGGAGAGCCAGCC
CCATCGGGGTTCCCCGCCGCCGGAAGCGGAAATAGCACCGGGCGCCGCCA
CAGTAGCTGTAACTGCCACCGCG

Various reporter genes are known in the art and may be used. Such reporter genes include but are not limited to fluorescent and luminescent proteins. In certain embodiments, the reporter gene is green fluorescent protein (GFP). In specific embodiments, the report gone plasmid construct comprises the sequence as set forth below.

5′GGCCTAACTGGCCGGTACCTGAGCTCGCTAGCCGTGTCTTCCTTGCCT
ACCAGCCTCACCTGATGGGCTCGTGTTCTCTCCGTCCCCGATCCACTCGG
GCTCCGGCAGCTGCTGCTTGGGCGCCTTCGGCATCGCGGTGGCAGAACTA
GAAACGAGTTACAGATAGAAACTAGAATATGCTTTTTAAAAAAACAAAAA
ACAAAACAAACAAAAAAACAGTATGCCTCAACTCCTTCATACTAGTAGGA
AATTATTATGTTCATTCCTTGAGTCTCGCGGCGTCGGGAGGTCACGGCGT
CAGGCTTCCCAGACAGTCGTAAACGCCATGTGTTTACGCGACTGGAGCAA
GCGGACGCCGGCCCCGCTCCGTCATTGCAGGCCACGCCTCCACTGAACCA
GGGCCACGCCCCCGAGATGACGGCGAAGCTCGCACGTGCGCAGCCCGGGG
GGGGGGTTGGCCGCGCCAGCTTGGAGAGCCAGCCCCATCGGGGTTCCCCG
CCGCCGGAAGCGGAAATAGCACCGGGCGCCGCCACAGTAGCTGTAACTGC
CACCGCGGCCACCATGGTGAGCAAGGGCGAGGAGCTGTTCACCGGGGTGG
TGCCCATCCTGGTCGAGCTGGACGGCGACGTAAACGGCCACAAGTTCAGC
GTGTCCGGCGAGGGCGAGGGCGATGCCACCTACGGCAAGCTGACCCTGAA
GTTCATCTGCACCACCGGCAAGCTGCCCGTGCCCTGGCCCACCCTCGTGA
CCACCCTGACCTACGGCGTGCAGTGCTTCAGCCGCTACCCCGACCACATG
AAGCAGCACGACTTCTTCAAGTCCGCCATGCCCGAAGGCTACGTCCAGGA
GCGCACCATCTTCTTCAAGGACGACGGCAACTACAAGACCCGCGCCGAGG
TGAAGTTCGAGGGCGACACCCTGGTGAACCGCATCGAGCTGAAGGGCATC
GACTTCAAGGAGGACGGCAACATCCTGGGGCACAAGCTGGAGTACAACTA
CAACAGCCACAACGTCTATATCATGGCCGACAAGCAGAAGAACGGCATCA
AGGTGAACTTCAAGATCCGCCACAACATCGAGGACGGCAGCGTGCAGCTC
GCCGACCACTACCAGCAGAACACCCCCATCGGCGACGGCCCCGTGCTGCT
GCCCGACAACCACTACCTGAGCACCCAGTCCGCCCTGAGCAAAGACCCCA
ACGAGAAGCGCGATCACATGGTCCTGCTGGAGTTCGTGACCGCCGCCGGG
ATCACTCTCGGCATGGACGAGCTGTACAAGTAGTAACCGGGAATTCCGCC
CCTCTCCCCCCCTAACGTTACTGGCCGAAGCCGCTTGGAATAAGGCCGGT
GTGCGTTTGTCTATATGTTATTTTCCACCATATTGCCGTCTTTTGGCAAT
GTGAGGGCCCGGAAACCTGGCCCTGTCTTCTTGACGAGCATTCCTAGGGG
TCTTTCCCCTCTCGCCAAAGGAATGCAAGGTCTGTTGAATGTCGTGAAGG
AAGCAGTTCCTCTGGAAGCTTCTTGAAGACAAACAACGTCTGTAGCGACC
CTTTGCAGGCAGCGGAACCCCCCACCTGGCGACAGGTGCCTCTGCGGCCA
AAAGCCACGTGTATAAGATACACCTGCAAAGGCGGCACAACCCCAGTGCC
ACGTTGTGAGTTGGATAGTTGTGGAAAGAGTCAAATGGCTCTCCTCAAGC
GTATTCAACAAGGGGCTGAAGGATGCCCAGAAGGTACCCCATTGTATGGG
ATCTGATCTGGGGCCTCGGTGCACATGCTTTACATGTGTTTAGTCGAGGT
TAAAAAAACGTCTAGGCCCCCCGAACCACGGGGACGTGGTTTTCCTTTGA
AAAACACGATGATAATAAGATCTGGCCTCGGCGGCCAAGCTTGGCAATCC
GGTACTGTTGGTAAAGCCACCATGAACTCCTTCTCCACAAGCGCCTTCGG
TCCAGTTGCCTTCTCCCTGGGCCTGCTCCTGGTGTTGCCTGCTGCCTTCC
CTGCCCCAGCCACCATGGGTCTTCACACTCGAAGATTTCGTTGGGGACTG
GCGACAGACAGCCGGCTACAACCTGGACCAAGTCCTTGAACAGGGAGGTG
TGTCCAGTTTGTTTCAGAATCTCGGGGTGTCCGTAACTCCGATCCAAAGG
ATTGTCCTGAGCGGTGAAAATGGGCTGAAGATCGACATCCATGTCATCAT
CCCGTATGAAGGTCTGAGCGGCGACCAAATGGGCCAGATCGAAAAAATTT
TTAAGGTGGTGTACCCTGTGGATGATCATCACTTTAAGGTGATCCTGCAC
TATGGCACACTGGTAATCGACGGGGTTACGCCGAACATGATCGACTATTT
CGGACGGCCGTATGAAGGCATCGCCGTGTTCGACGGCAAAAAGATCACTG
TAACAGGGACCCTGTGGAACGGCAACAAAATTATCGACGAGCGCCTGATC
AACCCCGACGGCTCCCTGCTGTTCCGAGTAACCATCAACGGAGTGACCGG
CTGGCGGCTGTGCGAACGCATTCTGGCGTAAGGCCGCGACTCTAGAGTCG
GGGCGGCCGGCCGCTTCGAGCAGACATGATAAGATACATTGATGAGTTTG
GACAAACCACAACTAGAATGCAGTGAAAAAAATGCTTTATTTGTGAAATT
TGTGATGCTATTGCTTTATTTGTAACCATTATAAGCTGCAATAAACAAGT
TAACAACAACAATTGCATTCATTTTATGTTTCAGGTTCAGGGGGAGGTGT
GGGAGGTTTTTTAAAGCAAGTAAAACCTCTACAAATGTGGTAAAATCGAT
AAGGATCCGTTTGCGTATTGGGCGCTCTTCCGCTGATCTGCGCAGCACCA
TGGCCTGAAATAACCTCTGAAAGAGGAACTTGGTTAGCTACCTTCTGAGG
CGGAAAGAACCAGCTGTGGAATGTGTGTCAGTTAGGGTGTGGAAAGTCCC
CAGGCTCCCCAGCAGGCAGAAGTATGCAAAGCATGCATCTCAATTAGTCA
GCAACCAGGTGTGGAAAGTCCCCAGGCTCCCCAGCAGGCAGAAGTATGCA
AAGCATGCATCTCAATTAGTCACTCGAGGCAACCATAGTCCCGCCCCTAA
CTCCGCCCATCCCGCCCCTAACTCCGCCCAGTTCCGCCCATTCTCCGCCC
CATGGCTGACTAATTTTTTTTATTTATGCAGAGGCCGAGGCCGCCTCTGC
CTCTGAGCTATTCCAGAAGTAGTGAGGAGGCTTTTTTGGAGGCCTAGGCT
TTTGCAAAAAGCTCGATTCTTCTGACACTAGCGCCACCATGAAGAAGCCC
GAACTCACCGCTACCAGCGTTGAAAAATTTCTCATCGAGAAGTTCGACAG
TGTGAGCGACCTGATGCAGTTGTCGGAGGGCGAAGAGAGCCGAGCCTTCA
GCTTCGATGTCGGCGGACGCGGCTATGTACTGCGGGTGAATAGCTGCGCT
GATGGCTTCTACAAAGACCGCTACGTGTACCGCCACTTCGCCAGCGCTGC
ACTACCCATCCCCGAAGTGTTGGACATCGGCGAGTTCAGCGAGAGCCTGA
CATACTGCATCAGTAGACGCGCCCAAGGCGTTACTCTCCAAGACCTCCCC
GAAACAGAGCTGCCTGCTGTGTTACAGCCTGTCGCCGAAGCTATGGATGC
TATTGCCGCCGCCGACCTCAGTCAAACCAGCGGCTTCGGCCCATTCGGGC
CCCAAGGCATCGGCCAGTACACAACCTGGCGGGATTTCATTTGCGCCATT
GCTGATCCCCATGTCTACCACTGGCAGACCGTGATGGACGACACCGTGTC
CGCCAGCGTAGCTCAAGCCCTGGACGAACTGATGCTGTGGGCCGAAGACT
GTCCCGAGGTGCGCCACCTCGTCCATGCCGACTTCGGCAGCAACAACGTC
CTGACCGACAACGGCCGCATCACCGCCGTAATCGACTGGTCCGAAGCTAT
GTTCGGGGACAGTCAGTACGAGGTGGCCAACATCTTCTTCTGGCGGCCCT
GGCTGGCTTGCATGGAGCAGCAGACTCGCTACTTCGAGCGCCGGCATCCC
GAGCTGGCCGGCAGCCCTCGTCTGCGAGCCTACATGCTGCGCATCGGCCT
GGATCAGCTCTACCAGAGCCTCGTGGACGGCAACTTCGACGATGCTGCCT
GGGCTCAAGGCCGCTGCGATGCCATCGTCCGCAGCGGGGCCGGCACCGTC
GGTCGCACACAAATCGCTCGCCGGAGCGCAGCCGTATGGACCGACGGCTG
CGTCGAGGTGCTGGCCGACAGCGGCAACCGCCGGCCCAGTACACGACCGC
GCGCTAAGGAGGTAGGTCGAGTTTAAACTCTAGAACCGGTCATGGCCGCA
ATAAAATATCTTTATTTTCATTACATCTGTGTGTTGGTTTTTTGTGTGTT
CGAACTAGATGCTGTCGACCGATGCCCTTGAGAGCCTTCAACCCAGTCAG
CTCCTTCCGGTGGGCGCGGGGCATGACTATCGTCGCCGCACTTATGACTG
TCTTCTTTATCATGCAACTCGTAGGACAGGTGCCGGCAGCGCTCTTCCGC
TTCCTCGCTCACTGACTCGCTGCGCTCGGTCGTTCGGCTGCGGCGAGCGG
TATCAGCTCACTCAAAGGCGGTAATACGGTTATCCACAGAATCAGGGGAT
AACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCC
TGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAA
GACACGACTTATCGCCACTGGCAGCAGCCACTGGTAACAGGATTAGCAGA
GCGAGGTATGTAGGCGGTGCTACAGAGTTCTTGAAGTGGTGGCCTAACTA
CGGCTACACTAGAAGAACAGTATTTGGTATCTGCGCTCTGCTGAAGCCAG
TTACCTTCGGAAAAAGAGTTGGTAGCTCTTGATCCGGCAAACAAACCACC
GCTGGTAGCGGTGGTTTTTTTGTTTGCAAGCAGCAGATTACGCGCAGAAA
AAAAGGATCTCAAGAAGATCCTTTGATCTTTTCTACGGGGTCTGACGCTC
AGTGGAACGAAAACTCACGTTAAGGGATTTTGGTCATGAGATTATCAAAA
AGGATCTTCACCTAGATCCTTTTAAATTAAAAATGAAGTTTTAAATCAAT
CTAAAGTATATATGAGTAAACTTGGTCTGACAGCGGCCGCAAATGCTAAA
CCACTGCAGTGGTTACCAGTGCTTGATCAGTGAGGCACCGATCTCAGCGA
TCTGCCTATTTCGTTCGTCCATAGTGGCCTGACTCCCCGTCGTGTAGATC
ACTACGATTCGTGAGGGCTTACCATCAGGCCCCAGCGCAGCAATGATGCC
GCGAGAGCCGCGTTCACCGGCCCCCGATTTGTCAGCAATGAACCAGCCAG
CAGGGAGGGCCGAGCGAAGAAGTGGTCCTGCTACTTTGTCCGCCTCCATC
CAGTCTATGAGCTGCTGTCGTGATGCTAGAGTAAGAAGTTCGCCAGTGAG
TAGTTTCCGAAGAGTTGTGGCCATTGCTACTGGCATCGTGGTATCACGCT
CGTCGTTCGGTATGGCTTCGTTCAACTCTGGTTCCCAGCGGTCAAGCCGG
GTCACATGATCACCCATATTATGAAGAAATGCAGTCAGCTCCTTAGGGCC
TCCGATCGTTGTCAGAAGTAAGTTGGCCGCGGTGTTGTCGCTCATGGTAA
TGGCAGCACTACACAATTCTCTTACCGTCATGCCATCCGTAAGATGCTTT
TCCGTGACCGGCGAGTACTCAACCAAGTCGTTTTGTGAGTAGTGTATACG
GCGACCAAGCTGCTCTTGCCCGGCGTCTATACGGGACAACACCGCGCCAC
ATAGCAGTACTTTGAAAGTGCTCATCATCGGGAATCGTTCTTCGGGGCGG
AAAGACTCAAGGATCTTGCCGCTATTGAGATCCAGTTCGATATAGCCCAC
TCTTGCACCCAGTTGATCTTCAGCATCTTTTACTTTCACCAGCGTTTCGG
GGTGTGCAAAAACAGGCAAGCAAAATGCCGCAAAGAAGGGAATGAGTGCG
ACACGAAAATGTTGGATGCTCATACTCGTCCTTTTTCAATATTATTGAAG
CATTTATCAGGGTTACTAGTACGTCTCTCAAGGATAAGTAAGTAATATTA
AGGTACGGGAGGTATTGGACAGGCCGCAATAAAATATCTTTATTTTCATT
ACATCTGTGTGTTGGTTTTTTGTGTGAATCGATAGTACTAACATACGCTC
TCCATCAAAACAAAACGAAACAAAACAAACTAGCAAAATAGGCTGTCCCC
AGTGCAAGTGCAGGTGCCAGAACATTTCTCT3′

In certain embodiments, the ABCF1 promoter reporter gene construct may be used in a high-throughput assay to screen for compounds and compositions that regulate the expression of ABCF1. In specific embodiments, the construct comprises the sequence encoding eGFP under the control of the ABCF1 promoter and changes in fluorescence is measured. A worker skilled in the art would readily appreciate that the construct may be used to screen for upregulators and downregulators of ABCF1 expression. In addition, the reported gene construct may be used to quantitate ABCF1 modulators by using appropriate standards.

In specific embodiments, there is provided a method of determining the amount of a psychedelic compound known to modulate ABCF1 expression in a sample, the method comprising contacting a cell comprising an ABCF1 reporter gene construct with the sample, comparing the expression of the reporter gene with a standard to determine the amount of psychedelic compound in the sample.

In specific embodiments, there is provided a method of determining the amount of a psilocybin in a sample, the method comprising contacting a cell comprising an ABCF1 reporter gene construct with the sample, comparing the expression of ABCF1 with a standard to determine the amount of psilocybin in the sample.

Cells, including but not limited macrophages such as RAW 264.7 cell line or the dendritic cell line DC2.1, comprising the ABCF1 promoter reporter gene construct may be used in such assays.

In certain embodiments, the amount of psychedelic is determined by comparing the expression of ABCF1 or a reporter gene under the control of the ABCF1 promoter obtained from contacting the sample with a cell capable of expressing ABCF1 or comprising the ABCF1 promoter reporter gene construct to expression of ABCF1 or a reporter gene under the control of the ABCF1 promoter from contacting known amounts of the psychedelic (standard curve) to a cell capable of expressing ABCF1 or comprising the ABCF1 promoter reporter gene construct.

The amount of psychedelic in any sample, including natural products and extracts thereof, may be determined using this method. In specific embodiments, the product is a mushroom extract.

Escitalopram, an antidepressant of the SSRI (selective serotonin receptor inhibitor) class, has been reported to influence anti-inflammatory pathways in patient populations and it was concluded that ABCF1 is Escitalopram's putative therapeutic target. Accordingly, in certain embodiments, there is provided bioassay screens which utilize ABCF1 to identify new drugs for treatment of MDD.

ABCF1 regulates M1 to M2 transitions. In certain embodiments, there is provided bioassay screens which utilize ABCF1 to identify new drugs/extracts for treating autoimmune and comorbid neuropsychiatric disorders. For example, the screens may be used to identify drugs/extracts that modulate an immune response, regulate inflammation and/or autoimmune disease.

In certain embodiments, the methods may be used to identify agents/extracts that modulate ABCF1 expression and therefore may be useful in the identification of drugs/extracts. In specific embodiments, a reporter gene is placed under the control of the ABCF1 promoter and the reporter gene product is measured (either qualitatively or quantitatively).

### EXAMPLES

### Example 1: Escitalopram Induces ABCF1 in a Macrophage Cell Line

RAW macrophages were plated at 1×10⁵ cells/well and cultured for 2 days. The cells were incubated with 0.3 mM Escitalopram for 1 hour, and then harvested for total RNA, which was extracted for real time RT-PCR specific for ABCF1 and IL-4. CT values were normalized with CT value for the housekeeping gene from the DMSO control. The difference in the expression after drug treatment is consistent with polarization towards an M2-like phenotype (data were consistent in 3 separate experiments).

### Example 2: The Effect of Psylocibin, Psylocin and their Analogs on ABCF1 Transcription in a Macrophage Cell Line

### Preparation of Cells:

1. Macrophage cell line RAW264.7 (ATCC) were grown to 80% confluency in growth media (DMEM+10% FBS+glutamine).
2. Dilutions of the drugs were made at desired final concentrations for a Dose response experiment. The concentrations' used for this experiment are: 10 nM, 100 nM, 500 nM for Psilocin, Psylocibin, 4-Acetoxy-N, N-dimthyltryptamine, O-Acetyl Psilocin Fumerate, and 4-acetoxyindole.
3. Untreated cells were used as negative control and Escitalopram at 0.3 mM was used as a positive control to activate ABCF1 expression for all the experiments.
   Analysis by qPCR:

Primers used:
GAPDH FP:
(SEQ ID NO: 1)
TGGATTTGGACGCATTGGTC
GAPDH RP:
(SEQ ID NO: 2)
TTTGCACTGGTACGTGTTGAT
ABCF1 FP:
(SEQ ID NO: 3)
AGAAAGCCCGAGTTGTGTTTG
ABCF1 RP:
(SEQ ID NO: 4)
GCCCCCTTGTAGTCGTTGATG

1. 2 hours post treatment with drugs, the reaction was stopped by removing the media with the drug. Cells were then collected and RNA was isolated from these.
2. After checking the quality of the RNA, cDNA was generated and qPCR was run with ABCF1 primers as the target gene and GAPDH as the house keeping gene.
3. Normalized against the expression level of GAPDH, the fold change expression level of ABCF1 was calculated and tabulated for all treatment conditions.

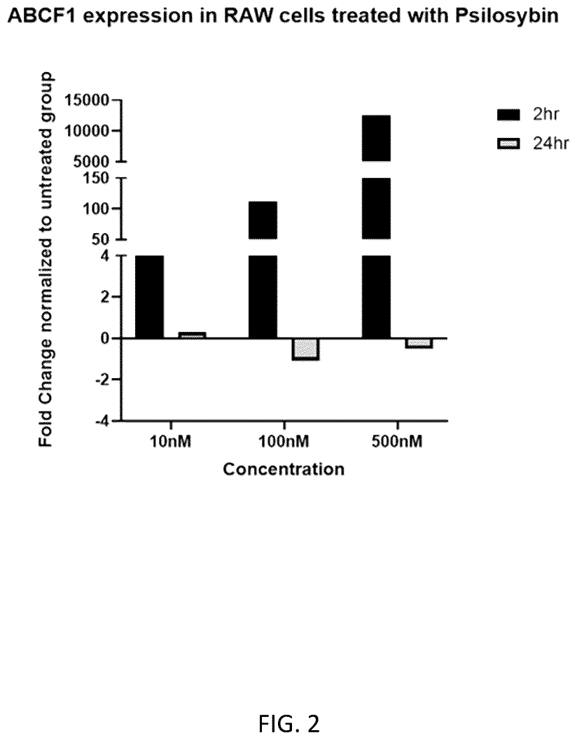

The results as set forth in FIG. 2 show psylocibin, psylocin and their analogs upregulate ABCF1 transcription.

### Example 3: Expression Levels of Markers Involved in Alzheimer's Disease Pathology in Murine Macrophage Cell Line, RAW264.7 Cells Post Treatment with Psychedelics

### Preparation of Cells:

1. Macrophage cell line RAW264.7 (ATCC) were grown to 80% confluency in growth media (DMEM+10% FBS+glutamine).
2. Confluent cells were treated with 10 uM of soluble amyloid beta (AB 1-16) for 2 hours or psychedelics at the prescribed concentrations at 2 hours post-stimulation or 24 or after restimualtion at 26 hours. Post treatment the cells were washed with PBS and cells were collected for RNA isolation followed by qRT-PCR to assess for expression levels of proteins involved in AD pathology; Hypoxia-inducible factor 1-alpha (Hif1-α), ATP Binding Cassette Subfamily F Member 1 (ABCF1).
   Analysis by qPCR:
3. Post treatment with amyloid beta at different time points, the reaction was stopped by removing the media with the drug. Cells were then collected and RNA was isolated from these cells.
4. After checking the quality of the RNA, cDNA was generated and qPCR was run with different target primers and GAPDH as the house keeping gene′
5. Normalized against the expression level of GAPDH, and further normalized against the expression level of untreated cells, the fold change expression level of different target genes was calculated and tabulated for all conditions.

### Results:

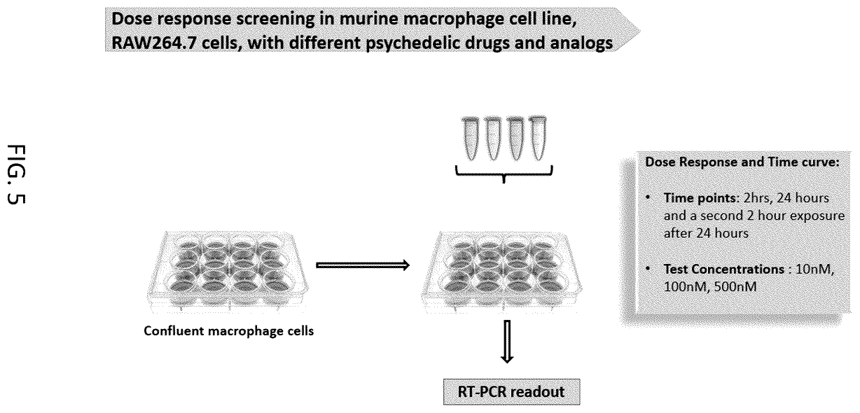

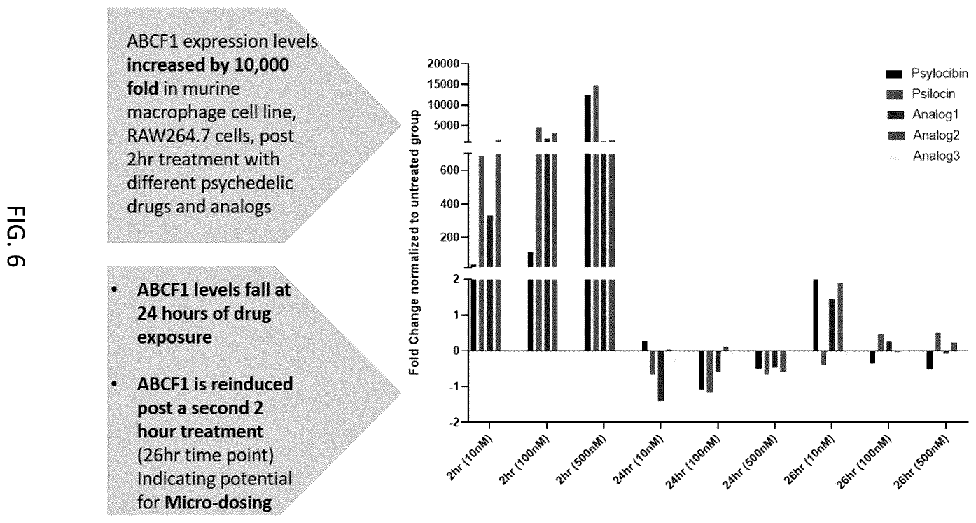

Expression of ABCF1 is shown in FIG. 5 to be modulated by treatment of RAW cells with psychedelics for 2 or 24 hours.
Expression of Hifα and ABCF1 are shown in FIG. 6 to be modulated by treatment of RAW cells with psychedelics for 2 hours.
The expression of ABCF1 is modulated by treatment of RAW cells with psychedelics for 2 hours incubation, 24 and after 2 hours of re-incubation the same cells for 2 hours 24-26 hours.

### Example 4: DC2.4 Cells Transfected with an ABCF1-GFP Vector

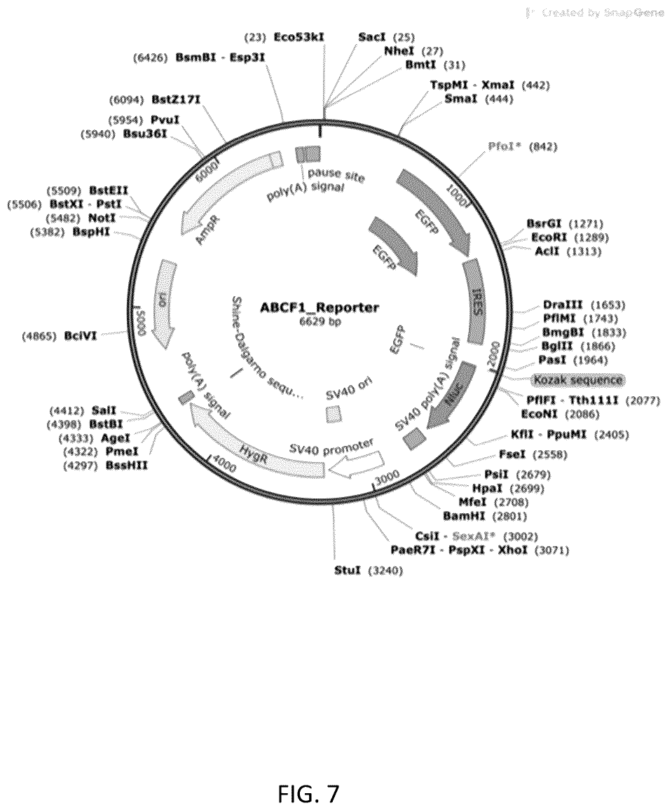

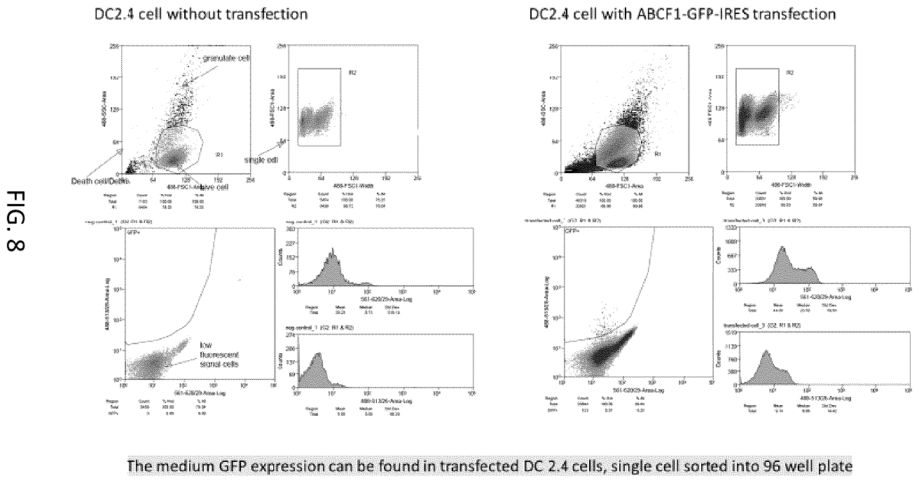

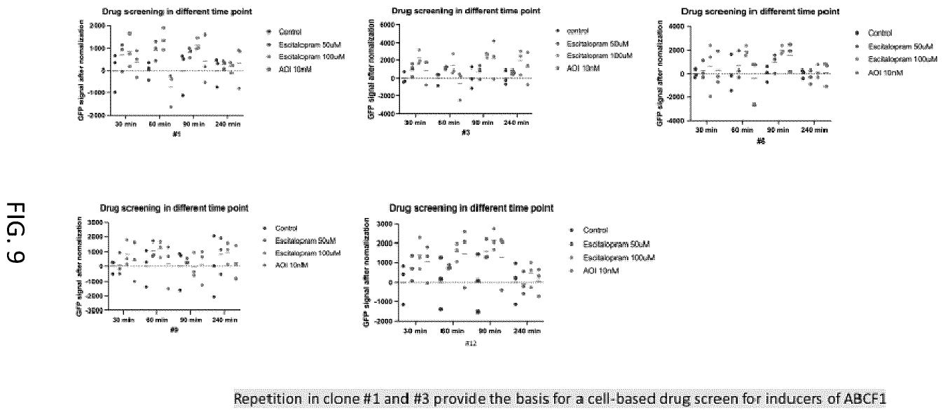

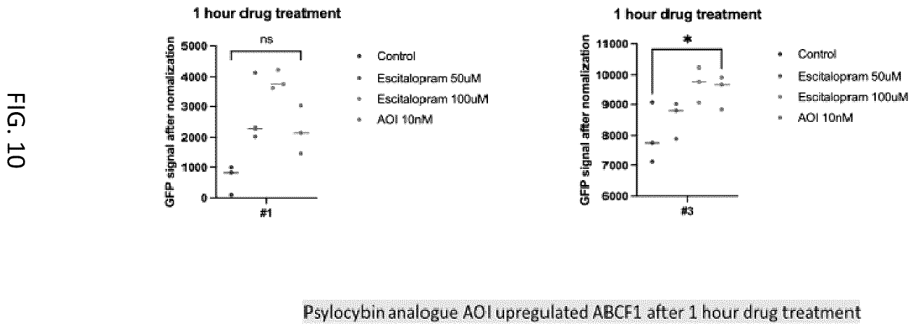

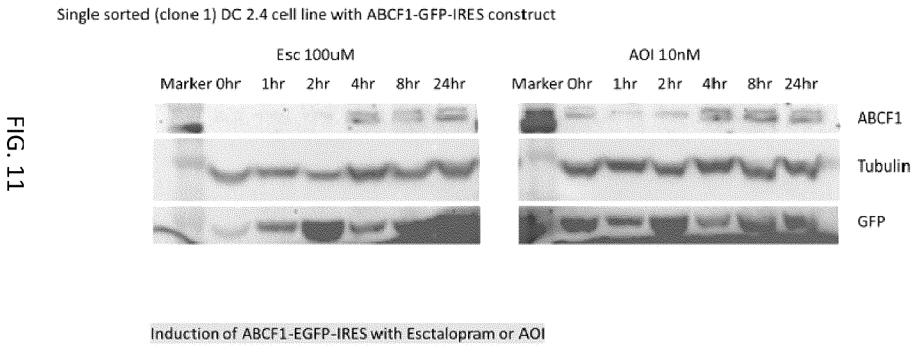

Cells for use in a high-throughput cell-based screening assay to screen for drugs and genes that stimulate ABCF1 expression were created. In particular, the cells comprise a construct comprising the ABCF1 promoter linked to the sequence encoding eGFP. In the assay, drugs/compositions may be screened for modulating expression of ABCF1 by measuring fluorescence.
1. DC2.4 cell line was used for the ABCF1-GFP vector (FIG. 7) transfection. DC 2.4 cell line was used as these cells have low endogenous ABCF1 expression.
2. After 3 days, based on the fluorescent signal, those transfected cells were single cell sorted using cell sorter, compared with DC2.4 cell line without transfection as a negative control. (Those cells with some fluorescent signals were transfected successfully as expected)
3. Those single cells were collected in a 96 well plate and grown around 2 weeks using DMEM+10% FBS+1% PS, and 12 colonies were picked.
4. Expended 12 colonies into 24 well plate to grow continuously around 2 weeks with DMEM+10% FBS+1% PS+100 nM Hygromycin (selection antibiotics which inserted in the vector) to keep the majority of cells which containing the vector.
5. 5 colonies were growing well and checked the colonies ABCF1 expression level (checked the GFP reporter in between negative control and positive control (50 uM and 100 μM) to see which colonies is the best to be drug screening cell).
See FIGS. 8 to 11.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

We claim:

1. A method of microdosing based on ABCF1 expression, the method comprising: a) determining ABCF1 expression in a sample obtained from a subject; b) administering a microdose of an agent which modulates ABCF1 expression if ABCF1 expression is less than a pre-determined amount; c) obtaining a further sample from the subject after a pre-determined amount of time and determining ABCF1 expression; d) administering a microdose of said agent if ABCF1 expression is less than a pre-determined amount; optionally repeating steps c) and d) one or more times.

2. The method of claim 1, wherein the agent is a psychedelic compound, natural product or extract.

3. The method of claim 1, wherein the agent is psilocybin or an analogue thereof.

4. A method of psilocybin microdosing based on ABCF1 expression, the method comprising: a) determining ABCF1 expression in a sample obtained from a subject; b) administering a microdose of psilocybin if ABCF1 expression is less than a pre-determined amount; c) obtaining a further sample from the subject after a pre-determined amount of time and determining ABCF1 expression; d) administering a microdose of said psilocybin if ABCF1 expression is less than a pre-determined amount; optionally repeating steps c) and d) one or more times.

5. A method of determining the amount of a psychedelic compound which modulates ABCF1 in a sample, the method comprising contacting a cell which is capable of expressing ABCF1 or a reporter gene under the control of the ABCF1 promoter with the sample, comparing the expression of ABCF1 or reporter with a standard to determine the amount of the compound in the sample.

6. The method of claim 5, wherein said sample is a sample of a natural product or extract thereof.

7. A method of determining the amount of a psilocybin in a sample, the method comprising contacting a cell which is capable of expressing ABCF1 or a reporter gene under the control of the ABCF1 promoter with the sample, comparing the expression of ABCF1 or reporter with a standard to determine the amount of psilocybini in the sample.

8. The method of any one of claims 5 to 7, wherein the amount is measured in international standard units.

9. The method of any one of claims 5 to 8, wherein ABCF1 mRNA is measured.

10. The method of any one of claims 5 to 8, wherein ABCF1 proteins including various protein isoforms and splice forms of ABCF1 are measured.

11. The method of claim 10, wherein an immunoassay using ABCF1 specific antibodies is used to measure ABCF1 proteins.

12. A method to identify agents that modulate ABCF1 expression, said method comprising contacting a cell expressing a reporter gene under the control of the ABCF1 promoter with the agent of interest; and measuring reporter gene product.


SEQUENCE LISTING (6 sequences)

SEQ ID NO: 1 - DNA, 20 bp, synthetic construct
  source 1..20: mol_type = other DNA
tggatttggacgcattggtc

SEQ ID NO: 2 - DNA, 21 bp, synthetic construct
  source 1..21: mol_type = other DNA
tttgcactggtacgtgttgat

SEQ ID NO: 3 - DNA, 21 bp, synthetic construct
  source 1..21: mol_type = other DNA
agaaagcccgagttgtgtttg

SEQ ID NO: 4 - DNA, 21 bp, synthetic construct
  source 1..21: mol_type = other DNA
gcccccttgtagtcgttgatg

SEQ ID NO: 5 - DNA, 523 bp, synthetic construct
  source 1..523: mol_type = other DNA
cgtgtcttccttgcctaccagcctcacctgatgggctcgtgttctctccgtccccgatccactcgggctccggcagctgctgcttgggcgccttcggcatcgcggtggcagaactagaaacgagttacagatagaaactagaatatgctttttaaaaaaacaaaaaacaaaacaaacaaaaaaacagtatgcctcaactccttcatactagtaggaaattattatgttcattccttgagtctcgcggcgtcgggaggtcacggcgtcaggcttcccagacagtcgtaaacgccatgtgtttacgcgactggagcaagcggacgccggccccgctccgtcattgcaggccacgcctccactgaaccagggccacgcccccgagatgacggcgaagctcgcacgtgcgcagcccgggggcggggttggccgcgccagcttggagagccagccccatcggggttccccgccgccggaagcggaaatagcaccgggcgccgccacagtagctgtaactgccaccgcg

SEQ ID NO: 6 - DNA, 6629 bp, synthetic construct
  source 1..6629: mol_type = other DNA
ggcctaactggccggtacctgagctcgctagccgtgtcttccttgcctaccagcctcacctgatgggctcgtgttctctccgtccccgatccactcgggctccggcagctgctgcttgggcgccttcggcatcgcggtggcagaactagaaacgagttacagatagaaactagaatatgctttttaaaaaaacaaaaaacaaaacaaacaaaaaaacagtatgcctcaactccttcatactagtaggaaattattatgttcattccttgagtctcgcggcgtcgggaggtcacggcgtcaggcttcccagacagtcgtaaacgccatgtgtttacgcgactggagcaagcggacgccggccccgctccgtcattgcaggccacgcctccactgaaccagggccacgcccccgagatgacggcgaagctcgcacgtgcgcagcccgggggcggggttggccgcgccagcttggagagccagccccatcggggttccccgccgccggaagcggaaatagcaccgggcgccgccacagtagctgtaactgccaccgcggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtaaccgggaattccgcccctctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataataagatctggcctcggcggccaagcttggcaatccggtactgttggtaaagccaccatgaactccttctccacaagcgccttcggtccagttgccttctccctgggcctgctcctggtgttgcctgctgccttccctgccccagccaccatgggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcactcgaggcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct